(19) United States
(12) Patent Application Publication
(10) Pub. No.: US 2018/0000732 A1
(43) Pub. Date: Jan. 4, 2018

(54) EMULSIONS FOR PARENTERAL ADMINISTRATION

(71) Applicant: Fresenius Kabi Deutschland GmbH, Bad Homburg (DE)

(72) Inventors: Edmundo Brito De La Fuente, Friedrichsdorf (DE); Getachew Assegehegn, Bad Homburg (DE); Crispulo Gallegos-Montes, Bad Homburg (DE); Telli Hekmatara, Frankfurt am Main (DE); Lida A. Quinchia-Bustamente, Bad Homburg (DE)

(21) Appl. No.: 15/545,503

(22) PCT Filed: Mar. 31, 2016
(86) PCT No.: PCT/EP2016/057130

§ 371 Date: Jul. 21, 2017

(30) Foreign Application Priority Data
Mar. 31, 2015 (EP) 15161846.9

(51) Int. Cl. A61K 9/107; A61K 9/00; A61K 47/44; A61K 47/46

(52) U.S. Cl. CPC A61K 9/107; A61K 47/46; A61K 9/0029; A61K 47/44

(57) ABSTRACT

A method for manufacturing oil-in-water emulsions for parenteral administration as well as to the use of such emulsions in the treatment or prevention of malnutrition and/or a deficiency in essential fatty acids and/or EPA and DHA and or stroke, sepsis, Alzheimer's disease or cancer.

### FIELD OF THE DISCLOSURE

The present disclosure relates to oil-in-water emulsions for parenteral administration.

The present disclosure further relates to a method for manufacturing the emulsion of the disclosure as well as to the use of the emulsion of the disclosure.

### BACKGROUND

Oil-in-water emulsions for parenteral administration have been used clinically for nutritional and medical purposes for several years. Various types of oils are used, of which soybean and safflawer oil were first introduced almost 50 years ago.

Oil-in-water emulsions for parenteral administration have to be sterile, pyrogen-free, well tolerated, isotonic or as close as possible to isotonicity, free of particulate impurities and storage stable. Their pH should be as close as possible to the pH of the blood. They may not contain more than 5% of oil droplets having a diameter of more than 5 μm. This parameter is referred to as “PFAT₅” and should not exceed 0.05 (compare USP 36 NF31<729>).

Commercially available products include e.g. Omegaven®, Intralipid®, SMOFlipid®, ClinOleic®, Lipofundin®, Lipoplus® and Lipidem®.

These are indicated for providing nutrition and—depending on the composition of the oil phase—for supplementing essential fatty acids and/or omega-3 fatty acids, e.g. docosahexaenoic acid (DHA) and eisosapentaenoic acid (EPA).

Further, health claims are directed to the improvement of the lung function, to the protection of the liver, to the reduction of the number of infections and organ complications, to the modulation of inflammatory processes, to the improvement of the oxygenation index as well as—unspecifically—to a general improvement of health, e.g. to the reduction of morbidity and mortality.

Especially emulsions comprising high proportions of the omega-3 fatty acids EPA and DHA are known to have beneficial effects, e.g. on the cardiovascular system, on cerebral function, as well as in combatting inflammatory conditions and reducing oxidative stress.

Most of the emulsions for parenteral administration contain egg yolk lecithin as an emulsifier which is most widely used in a concentration of 1.2 wt. % based on the total weight of the emulsion—irrespective of the lipid concentration.

The term lecithin refers to a complex mixture of acetone-insoluble phosphatides that mainly consists of phosphatidylcholine, phosphatidylethanolamine, phosphatidylserine, and phosphatidylinositol (jointly referred to as phospholipids), combined with various amounts of other substances such as triglycerides, fatty acids, and carbohydrates. The composition of lecithin (and hence also its physical properties) varies enormously depending on the source of the lecithin and the degree of purification. Egg yolk lecithin, for example, contains 69% phosphatidylcholine and 24% phosphatidylethanolamine, while soybean lecithin contains 21% phosphatidylcholine, 22% phosphatidylethanolamine and 19% phosphatidylinositol, along with other components. Lecithin has first been isolated from egg yolk. Its emulsifying properties mainly rest upon its high content in phospholipids.

Egg yolk lecithin is well tolerated and has long been found to be generally recognized as safe (GRAS).

However, the supply of egg yolk lecithin in the pharmaceutical grade suitable for parenteral administration is limited.

Further, frequent reports about viral infections of hens have led to an increasing public concern connected to the use of hen derived products.

Also, the quality of the lecithin may vary depending on the origin of the eggs.

Therefore it is desirable to provide emulsions comprising alternatives to egg yolk lecithin that at least equally well meet the complex requirements involved with the parenteral route of administration.

Krill oil has been suggested as such an alternative. Krill oil is an extract prepared from a species of antarctic krill, Euphausia superba. Krill oil comprises up to 50 wt. % phospholipids.

In WO2012/172411, krill oil is discussed for parenteral application.

The document relates to oil-in-water emulsions comprising krill oil as an emulsifier. The document discloses 40 g/l as an exemplary amount of krill oil.

The document further teaches emulsions comprising 2.4 wt. % krill oil as an emulsifier—assuming that the krill oil comprises as much as 50% phospholipids.

### SUMMARY

It has now been found that krill oil may be used as an emulsifier in surprisingly low concentrations allowing for egg lecithin free parenteral emulsions. In particular oil-in-water emulsions for parenteral administration comprising 0.5 to 2.2 wt. % krill oil based on the total weight of the emulsion may be free of egg yolk lecithin.

Further embodiments are set forth in the claims.

### DETAILED DESCRIPTION

The Krill Oil

Krill oil is an extract prepared from a species of antarctic krill, Euphausia superba. It has obtained GRAS (generally recognized as safe) status from the FDA and is commercially available, e.g. from Olympic Seafood (Bioriginal Europe/Asia B.V.) and Aker BioMarine

Antarctic AS. The emulsifying properties of krill oil mainly rely on its content in phospholipids (including phosphatidylcholine, phosphatidylethanolamine and phosphatidylinositol).

Krill oil usually comprises at most 50 wt. % phospholipids.

In case the krill oil comprises at most 50 wt. % phospholipids, according to the present disclosure it is used in concentrations of 0.5 to 2.2 wt. %, preferably 0.5 to 2.0 wt. %, based on the total weight of the emulsion. More preferably it is used in concentrations of 1.0 to 2.0 wt. % based on the total weight of the emulsion.

However, krill oil may also be obtained according to extraction methods allowing for phosholipid concentrations of more than 50 wt. % (see e.g. WO 2010/136900).

In case the krill oil comprises more than 50 wt. % phospholipids, according to the present disclosure it is used in concentrations of 0.5 to 2.2 wt. %, preferably 0.5 to 2.0 wt. % based on the total weight of the emulsion. More preferably it is used in concentration of 0.5 to 1.8 wt. % based on the total weight of the emulsion.Oil-in-water emulsion

The emulsion of the disclosure is an oil-in-water emulsion, i.e. the continous phase is aqueous and comprises oil droplets. The emulsion comprises the continous aqueous phase and preferably 2 wt. % to 30 wt. % of an oil phase based on the total weight of the emulsion. More preferably, the emulsion comprises 5 wt% to 30 wt. % of an oil phase based on the total weight of the emulsion, even more preferably 5 wt. % to 25 wt. % based on the total weight of the emulsion, most preferably 10 wt. % to 20 wt. % based on the total weight of th emulsion. For example, the emulsion comprises 10 wt. % or 20 wt. % of an oil phase based on the total weight of the emulsion.

The aqueous phase comprises water in purity suitable for parenteral administration, i.e. water for injection.

The Oil Phase

The oil phase may comprise a variety of different lipids, e.g. oils, e.g. soybean oil, olive oil, fish oil, fish oil extract, safflower oil, corn oil, sunflower oil, coconut oil, palm kernel oil, rapeseed oil, medium chain triglycerides (MCT) and mixtures thereof.

Preferably the oil phase comprises one or more oils selected from the group consisting of soybean oil, olive oil, fish oil, fish oil extract and MCT.

The term “fish oil” refers to purified fish oil and to purified—fish oil rich in omega 3 fatty acids” that according to the European Pharmacopoeia 6.0 comprises at least 9 wt. % of the omega-3-fatty acid docosahexaenoic acid (DHA) and at least 13 wt. % of the omega-3 fatty acid eisosapentaenoic acid (EPA) expressed as triglycerides.

The term “fish oil extract” refers to mixtures highly concentrated in EPA and DHA obtained e.g. from fish oil e.g. by supercritical fluid extraction and subsequent purification via e.g. chromatographic methods. Alternatively, the oil can be extracted using extraction techniques such as the one described in U.S. Pat. No. 6,750,048. Additional extraction and/or purification techniques are described in WO2001/076715 and WO2001/076385. The sum of EPA and DHA contained in these fish oil extracts is at least 500 milligram per gram of extract.

The fish oil extract comprises EPA and DHA in esterified form, e.g. in form of triglycerides or ethyl esters.

The term “medium chain triglycerides” refers to triglycerides of fatty acid being 6 to 12 carbon atoms in length, including caproic acid, caprylic acid, capric acid and lauric acid.

The antioxidant

The emulsion may comprise at least one pharmaceutically acceptable antioxidant.

An antioxidant useful in the emulsion of the disclosure may be any pharmaceutically acceptable compound having antioxidant activity, for example, the antioxidant may be selected form the group consisting of sodium metasulfite, sodium bisulfite, sodium sulfite, sodium thiosulfate, thioglycerol, thiosorbitol, thioglycolic acid, cysteine hydrochloride, n-acetly-cysteine, citric acid, alpha-tocopherol, beta-tocopherol, gamma-tocopherol, soluble forms of vitamin E, butylated hydroxyanisole (BHA), butylated hydroxytoluene (BHT), t-butylhydroquinone (TBHQ), monothioglycerol, propyl gallate, histidine, enzymes such as superoxide dismutase, catalase, selenium glutathione peroxidase, phospholipid hydroperoxide and glutathione peroxidase, Coenzyme Q10, tocotrienols, carotenoids, quinones, bioflavonoids, polyphenols, bilirubin, ascorbic acid, isoascorbic acid, uric acid, metal-binding proteins, ascorbic acid palmitate, an antioxidant obtained or obtainable from rosemary, rosemary extract and mixtures thereof.

The at least one antioxidant is in particular selected from the group consisting of alpha-tocopherol, beta-tocopherol, gamma-tocopherol, ascorbic acid, and mixtures of two or more thereof.

If present, the total amount of agents with antioxidant activity is preferably in the range of from 0.01 wt. % to 0.05 wt %, more preferably from 0.01 wt. % to 0.04 wt. %, more preferably from 0.01 wt. % to 0.03 wt. %, and even more preferably from 0.015 wt. % to 0.025 wt. %

based on the total weight of the emulsion.

The Tonicity Agent

The emulsion may comprise at least one pharmaceutically acceptable tonicity agent.

Tonicity agents are used to confer tonicity. Suitable tonicity agents may be selected from the group consisting of sodium chloride, mannitol, lactose, dextrose, sorbitol and glycerol.

Preferably, the tonicity agent is glycerol.

Preferably, the total amount of tonicity agents is in the range of 0.1 to 10 wt. %, more preferably from 1 wt. % to 5 wt. %, more preferably from 1 wt. % to 4 wt. %, more preferably 1 wt. % to 3 wt. %, more preferably from 1.5 wt. % to 2.8 wt. %, and even more preferably from 2.0 wt. % to 2.8 wt. % based on the total weight of the emulsion.

In case the tonicity agent is glycerol the most preferred amount is 2.0 wt. % to 2.5 wt. % based on the total weight of the emulsion.

Preferably, the emulsion has an osmolality in the range of 305 to 420 mOsmol/kg, measured with a Vapor Pressure Osmometer, Model 5520 (Vapro™) according to USP <785>.

pH Adjustment

The pH of the emulsion may be adjusted by adding solutions of conventionally known acids or bases such as HCl and NaOH or through the use of buffers, such as phosphate buffers.

The final pH of the emulsion is preferably in the range of from 6 to 9, more preferably between 7.5 and 8.5.

Preferably, the pH of the emulsion according to the disclosure is adjusted using a solution of NaOH.

The Co-Surfactant

The emulsion according to the disclosure may further comprise a pharmaceutically acceptable co-surfactant.

A co-surfactant is an amphiphilic molecule, i.e. a molecule that contains both hydrophilic and lipophilic groups. Usually, a co-surfactant substantially accumulates with the emulsifier at the interfacial layer. The hydrophile-lipophile balance (HLB) number is used as a measure of the ratio of hydrophilic and lipophilic groups present in a surfactant or co-surfactant, respectively. Usually, a co-surfactant with a very low HLB value (thus with a relatively high affinity to oil) is used together with a surfactant with a high HLB to modify the overall HLB of the system. Unlike the emulsifier, the co-surfactant may not be capable of forming self-associated structures, like micelles, on its own. Several kinds of molecules including nonionic emulsifiers, alcohols, amines and acids, can function as co-surfactants in a given system. The co-surfactant is usually used in a lower amount than that of the emulsifier. Apart from modifying the overall HLB value of the system, the co-surfactant has the effect of further reducing the interfacial tension and increasing the fluidity of the interface. Co-surfactants may also adjust the curvature of the interfacial film by partitioning between the tails of the emulsifier chains, allowing greater penetration of the oil between the emulsifier tails.

Preferably, the co-surfactant is a free unsaturated fatty acid or a salt thereof, preferably an omega-9 fatty acid or a salt thereof, more preferably a monounsaturated omega-9 fatty acid or a salt thereof, more preferably oleic acid or sodium oleate.

The total amount of the co-surfactant is preferably in the range of from 0.01 wt. % to 1 wt. %, more preferably in the range of from 0.02 wt. % to 0.5 wt. %, more preferably in the range of from 0.02 wt. % to 0.20 wt. % based on the total weight of the emulsion.

The Co-Solvent

The emulsion may further comprise a pharmaceutically acceptable co-solvent.

The term co-solvent refers to molecules that may increase the stability of the emulsion. In addition to making the environment more hydrophobic by reducing the dieelectric constant of water, co-solvents increase the amount of molecularly dispersed emulsifier and/or co-surfactant in the aqueous phase. Availability of free surfactant aids in the solubilization of hydrophobic molecules by creating pockets of hydrophobic regions within the aqueous phase.

The co-solvent may be selected from the group consisting of ethanol, propylene glycol and polyethylen glycol.

Preferably, the co-solvent is a polyalkylene glycol or an alkylene glycol, preferably polyethylen glycol or polypropylen glycol, more preferably polyethylene glycol (PEG).

The PEG preferably has a mean molecular weight in the range of from 100 to 20000 Da, more preferably in the range of from 200 to 1000 Da, more preferably in the range of from 300 to 600 Da, most preferably around 400 Da.

Preferably, the co-solvent is selected from the group consisting of PEG 200, PEG 300, PEG 400, PEG 600, PEG, 1000, PEG 1450, PEG 4000, PEG 6000, PEG 8000 and PEG 20000. Most preferably, the co-solvent is PEG 400.

Preferably, the total amount of co-solvents ranges from 0.1 wt % to 2.0 wt. %, more preferably from 0.25 wt. % to 1.75 wt. %, more preferably from 0.50 wt. % to 1.50 wt. %, more preferably from 0.70 wt. % to 1.40 wt. %, more preferably from 0.80 wt. % to 1.30 wt. %, and even more preferably from 0.90 wt. % to 1.20 wt. % based on the total weight of the emulsion.

The Droplet Size

Since the emulsion of the disclosure is an oil-in-water emulsion, the continous phase is aqueous and comprises oil droplets. These oil droplets are stabilized within the aqueous phase by at least one emulsifier and optionally further additives. The size of the oil droplets depends on the qualitative and quantitative composition of the emulsion and its preparation.

The oil droplets of the emulsion herein preferably have a mean diameter of 130 to 350 nm when measured directly upon sterilization using an LS 13 320 Laser Diffraction Particle Size Analyser (Beckman Coulter) according to USP <729>.

Preparation of the Emulsion

The present disclosure also relates to a method for preparing an emulsion for parenteral administration and to an emulsion obtained or obtainable by said method the emulsion comprising 0.5 wt. % to 2.2 wt. % krill oil based on the total weight of the emulsion, wherein the method comprises
a) providing an oil phase comprising the krill oil and optionally one or more lipids and/or at least one pharmaceutically acceptable antioxidant and/or a pharmaceutically acceptable co-surfactant,
b) providing an aqueous phase comprising water for injection and optionally a pharmaceutically acceptable tonicity agent and/or an agent for pH adjustment and/or a pharmaceutically acceptable co-surfactant and or a pharmaceutically acceptable co-solvent,
c) forming a pre-emulsion by mixing the oil phase provided in step a) with the aqueous phase provided in step b);
d) forming the emulsion by high-pressure homogenizing the pre-emulsion obtained in step c) and
e) sterilizing the emulsion obtained in step d),

It is to be understood that any of the optional further components of the emulsion may be added in any of the steps a), b), c) or d) or in one or more additional steps.

Step a)

Step a) is preferably carried out by mixing the krill oil with one or more oils selected from the group consisting of fish oil, fish oil extract, olive oil, soybean oil and MCT, and optionally the at least one antioxidant and/or the co-surfactant. This step is preferably carried out at a temperature of 50 to 65° C., wherein during this step the temperature may be varied or held essentially constant for a maximum 30 minutes until a homogeneous and clear phase is obtained.

It is to be understood that in step a) further additives may be added.

Step b)

Step b) is preferably carried providing water for injection and optionally adding the tonicity agent and/or the co-surfactant.

The aqueous phase is then heated to a temperature of 55 to 80° C., preferably for a time of 1 minute to 1 hour, more preferably from 5 to 30 minutes, more preferably from 5 to 15 minutes.

Preferably, step b) further comprises adjusting the pH to values between 7 and 10, preferably to a pH between 8 and 9, preferably by adding a solution of NaOH.

It is to be understood that in step b) further additives may be added.

Step c)

The method further comprises mixing the oil phase provided in step a) with the aqueous phase provided in step b) thereby forming a pre-emulsion. The mixing may be carried out by any method known to those skilled in the art. Preferably, the mixing is carried out using a high shear mixer.

Preferably the oil phase is added to the aqueous phase or vice-versa at a temperature in the range of from 50 to 80° C.

Preferably the oil phase is added to the aqueous phase or vice-versa at a pressure such as as under nitrogen pressure, in the range of from 0.20 to 0.80 bar, more preferably from 0.2 to 0.4 bar. During this step the pressure may be varied or held essentially constant.

According to a preferred embodiment, the mixture is stirred for a time in the range of from 1 minute to 1 hour, preferably from 10 to 30 minutes. During this step, the temperature may be varied or held essentially constant.

It is to be understood that further components may also be added after the formation of the pre-emulsion. According to a preferred embodiment, the pH of the pre-emulsion is adjusted to a pH in the range of from 8 to 10, in particular by adding sodium hydroxide, if necessary.

Step d)

The method further comprises the homogenization of the pre-emulsion obtained in step c). This homogenization may be carried out by any suitable method known to those skilled in the art.

Preferably the mixture is homogenized at a temperature in the range of from 40 to 70° C., preferably from 40 to 60° C., more preferably from 50 to 60° C.

Preferably, the pre-emulsion is homogenized at a pressure in the range of from 400 to 600 bar, more preferably from 450 to 550 bar. During this step the pressure may be varied or held essentially constant.

Preferably, the homogenization is carried out using a high pressure homogenizer or a microfluidizer.

Step e)

The method further comprises the sterilization of the emulsion obtained in step d) to ensure its suitability for parenteral administration.

The sterilization may be carried out by any suitable method known to those skilled in the art. In particular, the sterilization is carried out by autoclaving, preferably at a temperature in the range of from 119 to 122° C., more preferably at a temperature around 121° C., preferably for a time in the range of from 1 minute to 30 minutes, preferably of from 10 minutes to 15 minutes.

Packaging of the Emulsion

The emulsion according to the disclosure may be comprised in a suitable container.

The container may be made of any suitable material substantially inert against the ingredients of the emulsion according to the disclosure, preferably even upon sterilization. It may have any suitable form, e.g. the form of a bottle, a bag or a syringe. The material may for example comprise glass or plastic. The plastic material may comprise one or more polymers and optionally further additives.

In one embodiment the container is a glass bottle, preferably a transparent glass bottle.

In another embodiment the container is a plastic bag, preferably a transparent plastic bag.

In one embodiment the plastic material comprises 3 layers. The first layer, also referred to as the inner layer, is in direct contact with the emulsion according to the disclosure. The second layer, also referred to as the middle layer, and the third layer, also referred to as the outer layer, are not in direct contact with the emulsion.

Preferably, the middle layer is thicker than the inner and the outer layer, providing for requisite stability.

Preferably, the inner, the middle and the outer layer all comprise a thermoplastic elastomer (TPE), wherein preferably, the content in TPE is highest in the middle layer, warranting the required flexibility.

The inner layer, in addition to the TPE, preferably comprises a polyolefine co-polymer.

Preferably, the polyolefine co-polymer comprises a polypropylene-polyethylene co-polymer.

Preferably, the TPE is a styrenic block co-polymer, more preferably Styrene-Ethylen-Butylen-Styrene (SEBS).

The inner layer preferably comprises 70 to 90 wt. % of the polyolefine co-polymer and 10 to 30 wt. % of the TPE.

Preferably, the inner layer has a thickness of 10 to 90 μm, more preferably 10 to 70 μm, even more preferably 10 to 50 μm, most preferably 20 to 40 μm.

The middle layer, in addition to the TPE, preferably comprises a polyolefine co-polymer.

Preferably, the polyolefine co-polymer comprises a polypropylene-polyethylene co-polymer.

Preferably, the TPE comprises a styrenic block co-polymer, more preferably 2 styrenic block co-polymers, most preferably Styrene-Ethylen-Butylen-Styrene (SEBS) and Styrene-Isopren-Styrene (SIS).

The middle layer preferably comprises 40 to 70 wt. %, more preferably 50 to 60 wt. % of the polyolefine co-polymer and 30 to 60 wt. %, more preferably 40 to 50 wt. % of the TPE.

Preferably, the middle layer has a thickness of 30 to 200 μm, more preferably 50 to 190, even more preferably 70 to 180 μm, most preferably 100 to 150 μm.

The outer layer, in addition to the TPE preferably comprises a polyolefine. Preferably, the polyolefine comprises polypropylene.

Preferably, the TPE is a styrenic block co-polymer, preferably Styrene-Ethylen-Butylen-Styrene (SEBS).

The outer layer preferably comprises 70 to 95 wt. %, more preferably 80 to 90 wt. %, of the polyolefine and 5 to 30 wt. %, more preferably 10 to 20 wt. %, of the TPE.

Preferably, the outer layer has a thickness of 5 to 50 μm, more preferably 10 to 50, even more preferably 15 to 45 μm, most preferably 20 to 40 μm.

The container may optionally further be comprised in an overpouch. The overpouch may comprise several layers comprised of different materials. Preferably, the overpouch is transparent and/or impermeable to oxygen.

The Present Disclosure Includes Inter Alia the Following Aspects:

In a first aspect the present disclosure relates to an oil-in-water emulsion comprising 0.5 to 2.2 wt. %, preferably 0.5 to 2.0 wt. % krill oil based on the total weight of the emulsion, wherein the emulsion is free of egg yolk lecithin.

In a second aspect the present disclosure relates to an emulsion according to aspect 1 comprising 2 to 30% of an oil phase based on the total weight of the emulsion.

In a third aspect the present disclosure relates to an emulsion according to aspect 1 or 2, wherein the oil phase comprises one or more oils selected from the group consisting of soybean oil, olive oil, fish oil, fish oil extract, safflower oil, corn oil, sunflower oil, coconut oil, palm kernel oil, rapeseed oil and medium chain triglycerides (MCT).

In a fourth aspect the present disclosure relates to an emulsion according any of the preceding aspects, wherein the oil phase comprises soybean oil.

In a fifth aspect the present disclosure relates to an emulsion according to any of the preceding aspects, wherein the oil phase comprises fish oil.

In a sixth aspect the present disclosure relates to an emulsion according to any of the preceding aspects, wherein the oil phase comprises fish oil extract.

In a seventh aspect the present disclosure relates to an emulsion according to any of the preceding aspects, wherein the oil phase comprises fish oil, MCT, soybean oil and olive oil.

The emulsion according to the disclosure may further comprise at least one pharmaceutically acceptable antioxidant, e.g. alpha-tocopherol or a mixture of alpha-, beta, gamma-and delta-tocopherols.

Preferably, the emulsion according to the disclosure comprises a pharmaceutically acceptable tonicity agent, preferably glycerol.

Preferably, the emulsion according to the disclosure comprises an agent for pH adjustment, preferably NaOH.

The emulsion according to the disclosure may further comprise a co-surfactant, preferably oleic acid or sodium oleate.

The emulsion according to the disclosure may further comprise a co-solvent, preferably polyethylen glycol (PEG).

The oil droplets of the emulsion according to the disclosure preferably have a mean diameter of 150 to 350 nm when measured directly upon sterilization.

In an eighth aspect the present disclosure relates to a dose unit of the emulsion according to any of the aspects 1 to 7, wherein the dose unit comprises 50 to 500 ml, e.g. 50 ml, 100 ml, 250 ml or 500 ml, of the emulsion.

In an ninth aspect the present disclosure relates to a method for manufacturing the emulsion according to the disclosure, comprising
a) providing an oil phase comprising the krill oil and optionally one or more oils selected from the group according to any of the preceding aspects and/or at least one pharmaceutically acceptable antioxidant and/or a pharmaceutically acceptable co-surfactant;
b) providing an aqueous phase comprising water for injection and optionally a pharmaceutically acceptable tonicity agent and/or an agent for pH adjustment and/or a pharmaceutically acceptable co-surfactant and or a pharmaceutically acceptable co-solvent;
c) forming a pre-emulsion by mixing the oil phase provided in step a) with the aqueous phase provided in step b);
d) forming the emulsion by high-pressure homogenizing the pre-emulsion obtained in step c) and
e) sterilizing the emulsion obtained in step d),
wherein the emulsion is optionally filled into a suitable container either before or after sterilizing the emulsion.

In a tenth aspect the present disclosure relates to the emulsion or dose unit according to any of the aspects 1 to 8 or obtained by the method of aspect 9 for use in the treatment or prevention of a medical condition.

In a eleventh aspect the present disclosure relates to the emulsion or the dose unit according to any of the aspects 1 to 8 or obtained by the method of aspect 9 for use in treating or preventing a deficiency in essential fatty acids and/or EPA and DHA and/or malnutrition.

In an twelfth aspect the present disclosure relates to the emulsion or dose unit according to any of the aspects 1 to 8 or obtained by the method of aspect 9 for use in treating or preventing stroke, sepsis, Alzheimer's disease or cancer.

In a thirteenth aspect the present disclosure relates to the emulsion or dose unit according to any of the aspects 1 to 8 or obtained by the method of aspect 9 for use in treating or preventing a deficiency in essential fatty acids and/or EPA and DHA and/or malnutrition in an individual suffering from or being at risk of stroke, sepsis, Alzheimer's disease or cancer.

In a fourteenth aspect the present disclosure relates to the use of the emulsion or dose unit according to any of the aspects 1 to 8 or obtained by the method of aspect 9 for providing nutrition to an individual and/or for supplementing an individual with essential fatty acids and/or EPA and DHA.

In a fifteenth aspect the present disclosure relates to the use of the emulsion or dose unit according to any of the aspects 1 to 8 or obtained by the method of aspect 9 for providing nutrition to an individual and/or for supplementing an individual with essential fatty acids and/or EPA and DHA, wherein the individual is suffering from or at risk of stroke, sepsis, Alzheimer's disease or cancer.

In a sixteenth aspect the present disclosure relates to a container comprising the emulsion according to any of the aspects 1 to 7 or the dose unit according to aspect 8, wherein the container material is glass or a plastic, preferably wherein the container is a glass bottle or a plastic bag.

In a seventeenth aspect the present disclosure relates to a container according to aspect 16, wherein the plastic material comprises 3 layers.

Preferably, the inner layer comprises a polyolefine co-polymer and a thermoplastic elastomer.

Preferably, the inner layer comprises 70 to 90 wt. % of a polyolefine co-polymer and 10 to 30 wt. % of a thermoplastic elastomer.

Preferably, the thermoplastic elastomer is a styrenic block co-polymer, preferably Styrene-Ethylen-Butylen-Styrene (SEBS).

Preferably, the polyolefine co-polymer is a polypropylene-polyethylene co-polymer. Preferably, the inner layer has a thickness of 10 to 90, more preferably 10 to 70, even more preferably 10 to 50, most preferably 20 to 40 μm.

### Embodiments

1) Oil-in-water emulsion for parenteral administration comprising 0.5 to 2.2 wt. %, preferably 0.5 to 2.0 wt. % krill oil based on the total weight of the emulsion, wherein the emulsion is free of egg yolk lecithin.
2) Emulsion according to embodiment 1 comprising 0.5 to 2.0 wt. %, preferably 0.5 to 1.8 wt. % krill oil, wherein the krill oil comprises more than 50 wt. % phospholipids.
3) Emulsion according to embodiment 1 comprising 0.5 to 2.0 wt. %, preferably 1.0 to 2.0 wt. % krill oil, wherein the krill oil comprises at most 50 wt. % phospholipids.
4) Emulsion according to any of the preceding embodiments comprising 2 to 30 wt. % of an oil phase based on the total weight of the emulsion.
5) Emulsion according to any of the preceding embodiments, wherein the oil phase comprises one or more oils selected from the group consisting of soybean oil, olive oil, fish oil, fish oil extract, safflower oil, corn oil, sunflower oil, coconut oil, palm kernel oil, rapeseed oil and medium chain triglycerides (MCT).
6) Emulsion according to any of the preceding embodiments, wherein the oil phase comprises fish oil.
7) Emulsion according to any of the preceding embodiments, wherein the oil phase comprises fish oil extract.
8) Emulsion according to any of the preceding embodiments, wherein the oil phase comprises olive oil.
9) Emulsion according to any of the preceding embodiments, wherein the oil phase comprises soybean oil.
10) Emulsion according to any of the preceding embodiments, wherein the oil phase comprises MCT.
11) Emulsion according to any of the preceding embodiments, wherein the oil phase comprises fish oil, soybean oil, olive oil, and medium chain triglycerides (MCT).
12) Emulsion according to any of the preceding embodiments, wherein the emulsion further comprises a pharmaceutically acceptable tonicity agent, preferably glycerol.
13) Emulsion according to any of the preceding embodiments, wherein the emulsion further comprises an agent for pH adjustment, preferably NaOH.
14) Emulsion according to any of the preceding embodiments, wherein the emulsion further comprises at least one pharmaceutically acceptable antioxidant, preferably one or more selected from the group consisting of alpha, beta, gamma and delta tocopherols.
15) Emulsion according to any of the preceding embodiments, wherein the emulsion further comprises a pharmaceutically acceptable co-surfactant, preferably oleic acid or sodium oleate.
16) Emulsion according to any of the preceding embodiments, wherein the emulsion further comprises a pharmaceutically acceptable co-solvent, preferably PEG.
17) Emulsion according to any of the preceding embodiments, wherein the oil droplets comprised in the oil-in-water emulsion have a mean diameter of 130 to 350 nm.
18) Dose unit comprising 50 to 500 ml of the emulsion according to any of the embodiments 1 to 17.
19) Dose unit according to embodiment 18 comprising 50 ml, 100 ml, 250 ml, or 500 ml of the emulsion according to any of embodiments 1 to 17.
20) Emulsion or dose unit according to any of the preceding embodiments for use in treating or preventing a deficiency in essential fatty acids.
21) Emulsion or dose unit according to any of embodiments 1 to 19 for use in treating or preventing a deficiency in EPA and DHA.
22) Emulsion or dose unit according to any of embodiments 1 to 19 for use in treating or preventing a deficiency in essential fatty acids, EPA and DHA.
23) Emulsion or dose unit according to any embodiments 1 to 19 for use in treating or preventing malnutrition.
24) Emulsion or dose unit according to any of embodiments 1 to 19 for use in treating or preventing malnutrition and a deficiency in EPA and DHA.
25) Emulsion or dose unit according to any of embodiments 1 to 19 for use in treating or preventing malnutrition and a deficiency in essential fatty acids, EPA and DHA.
26) Emulsion or dose unit according to any of embodiments 1 to 19 for use as a medicament, in particular for use in treating or preventing stroke, sepsis, Alzheimer's disease or cancer.
27) Emulsion or dose unit according to any of embodiments 1 to 19 for use in treating or preventing a deficiency in essential fatty acids in a patient suffering from or being at risk of stroke, sepsis, Alzheimer's disease or cancer.
28) Emulsion or dose unit according to any of embodiments 1 to 19 for use in treating or preventing a deficiency in EPA and DHA in a patient suffering from or being at risk of stroke, sepsis, Alzheimer's disease or cancer.
29) Emulsion or dose unit according to any of embodiments 1 to 19 for use in treating or preventing a deficiency in essential fatty acids, EPA and DHA in a patient suffering from or being at risk of stroke, sepsis, Alzheimer's disease or cancer.
30) Emulsion or dose unit according to any of embodiments 1 to 19 for use in treating or preventing malnutrition in a patient suffering from or being at risk of stroke, sepsis, Alzheimer's disease or cancer.
31) Emulsion or dose unit according to any of embodiments 1 to 19 for use in treating or preventing malnutrition and a deficiency in essential fatty acids in a patient suffering from or being at risk of stroke, sepsis, Alzheimer's disease or cancer.
32) Emulsion or dose unit according to any of embodiments 1 to 19 for use in treating or preventing malnutrition and a deficiency in EPA and DHA in a patient suffering from or being at risk of stroke, sepsis, Alzheimer's disease or cancer.
33) Emulsion or dose unit according to any of embodiments 1 to 19 for use in treating or preventing malnutrition and a deficiency in essential fatty acids, EPA and DHA in a patient suffering from or being at risk of stroke, sepsis, Alzheimer's disease or cancer.
34) Method for treating or preventing stroke, sepsis, Alzheimer's disease or cancer comprising the parenteral administration of an emulsion or a dose unit according to any of embodiments 1 to 19.
35) Method for treating or preventing a deficiency in essential fatty acids comprising the parenteral administration of an emulsion or a dose unit according to any of embodiments 1 to 19.
36) Method for treating or preventing a deficiency in EPA and DHA comprising the parenteral administration of an emulsion or a dose unit according to any of embodiments 1 to 19.
37) Method for treating or preventing a deficiency in essential fatty acids, EPA and DHA comprising the parenteral administration of an emulsion or a dose unit according to any of embodiments 1 to 19.
38) Method for treating or preventing malnutrition comprising the parenteral administration of an emulsion or a dose unit according to any of embodiments 1 to 19.
39) Method for treating or preventing malnutrition and a deficiency in essential fatty acids comprising the parenteral administration of an emulsion or a dose unit according to any of embodiments 1 to 19.
40) Method for treating or preventing malnutrition and a deficiency in EPA and DHA comprising the parenteral administration of an emulsion or a dose unit according to any of embodiments 1 to 19.
41) Method for treating or preventing malnutrition and a deficiency in essential fatty acids, EPA and DHA comprising the parenteral administration of an emulsion or a dose unit according to any of embodiments 1 to 19.
42) Method according to any of embodiments 32 to 39, wherein the emulsion or dose unit is administered to an individual suffering from or being at risk of stroke, sepsis, Alzheimer's disease or cancer.
43) Use of the emulsion or dose unit according to any of embodiments 1 to 19 for providing parenteral nutrition to an individual.
44) Use of the emulsion or dose unit according to any of embodiments 1 to 19 for supplementing an individual with essential fatty acids.
45) Use of the emulsion or dose unit according to any of embodiments 1 to 19 for supplementing an individual with EPA and DHA.
46) Use of the emulsion or dose unit according to any of embodiments 1 to 19 for supplementing an individual with essential fatty acids, DHA and EPA.
47) Use of the emulsion or dose unit according to any of embodiments 1 to 19 for providing parenteral nutrition to an individual and for supplementing the individual with EPA and DHA.
48) Use of the emulsion or dose unit according to any of embodiments 1 to 19 for providing parenteral nutrition to an individual and for supplementing the individual with essential fatty acids.
49) Use of the emulsion or dose unit according to any of embodiments 1 to 19 for providing parenteral nutrition to an individual and for supplementing the individual with essential fatty acids, EPA and DHA.
50) Use according to any of embodiments 43 to 49, wherein the individual is suffering from or being at risk of stroke, sepsis, Alzheimer's disease or cancer.
51) Method for manufacturing an emulsion according to any of embodiments 1 to 17, comprising
   a) providing an oil phase comprising the krill oil and optionally one or more oils selected from the group according to any of the embodiments 5 to 11 and/or at least one pharmaceutically acceptable antioxidant and/or a pharmaceutically acceptable co-surfactant,
   b) providing an aqueous phase comprising water for injection and optionally a pharmaceutically acceptable tonicity agent and/or an agent for pH adjustment and/or a pharmaceutically acceptable co-surfactant and or a pharmaceutically acceptable co-solvent,
   c) forming a pre-emulsion by mixing the oil phase provided in step a) with the aqueous phase provided in step b);
   d) forming the emulsion by high-pressure homogenizing the pre-emulsion obtained in step c) and
   e) sterilizing the emulsion obtained in step d)
52) Method according to embodiment 49, wherein step a) is conducted by mixing the components of the oil phase at a temperature of 50 to 65° C.
53) Method according to embodiment 47 or 48, wherein step b) is conducted by mixing the components of the aqueous phase at a temperature of 55 to 80° C.
54) Container comprising the emulsion or dose unit according to any of the embodiments 1 to 19, wherein the container material comprises glass or plastic.
55) Container according to embodiment 54 being a bottle, a bag or a syringe, preferably a glass bottle or a plastic bag.
56) Container according to embodiment 54 or 55, wherein the plastic material comprises 3 layers, wherein preferably the middle layer is thicker than the inner and the outer layer and/or wherein all layers comprise a thermoplastic elastomer and/or wherein the content of thermoplastic elastomer is highest in the middle layer.
57) Container according to embodiment 55, wherein the inner layer, being in direct contact with the emulsion, comprises a polyolefine co-polymer and a thermoplastic elastomer.
58) Container according to any of the embodiments 55 to 57, wherein the inner layer comprises 70 to 90 wt. % of a polyolefine co-polymer and 10 to 30 wt. % of a thermoplastic elastomer.
59) Container according to embodiment 57 or 58, wherein the thermoplastic elastomer comprises a styrenic block co-polymer.
60) Container according to any of the embodiments 57 to 59, wherein the thermoplastic elastomer comprises Styrene-Ethylen-Butylen-Styrene.
61) Container according to any of the embodiments 57 to 60, wherein the polyolefine co-polymer is a polypropylene-polyethylene co-polymer.
62) Container according to any of the embodiments 56 to 61, wherein the inner layer has a thickness of 10 to 90, preferably 10 to 70, more preferably 10 to 50, most preferably 20 to 40 μm.
63) Container according to any of the embodiments 56 to 62, wherein the middle layer, not being in direct contact with the emulsion, comprises a polyolefine co-polymer and at least one thermoplastic elastomer.
64) Container according to any of the embodiments 56 to 63, wherein the middle layer comprises 40 to 70, preferably 50 to 60 wt. % of a polyolefine co-polymer and 30 to 60, preferably 40 to 50 wt. % of at least one thermoplastic elastomer.
65) Container according to embodiment 63 or 64, wherein the polyolefine co-polymer comprises a polypropylene-polyethylene co-polymer.
66) Container according to any of the embodiments 63 to 65, wherein the at least one thermoplastic elastomer comprises at least one styrenic block co-polymer, preferably two styrenic block co-polymers.
67) Container according to any of the embodiments 63 to 66, wherein the at least one thermoplastic elastomer comprises Styrene-Ethylen-Butylen-Styrene and Styrene-Isopren-Styrene.
68) Container according to any of the embodiments 56 to 67, wherein the middle layer has a thickness of 30 to 200, preferably 50 to 190, more preferably 70 to 180, most preferably 100 to 150 μm.
69) Container according to any of the embodiments 56 to 68, wherein the outer layer, not being in direct contact with the emulsion, comprises a polyolefine co-polymer and a thermoplastic elastomer.
70) Container according to any of the embodiments 56 to 69, wherein the outer layer, comprises 70 to 95, preferably 80 to 90 wt. % of a polyolefine co-polymer and 5 to 30, preferably 10 to 20 wt. % of a thermoplastic elastomer.
71) Container according to embodiment 69 or 70, wherein the polyolefine comprises polypropylene and the thermoplastic elastomer comprises Styrene-Ethylen-Butylen-Styrene.
72) Container according to any of the embodiments 56 to 71, wherein the third layer has a thickness of 5 to 50, preferably, 10 to 50, more preferably 15 to 45, most preferably 20 to 40 μm.
73) Container according to any of the embodiments 54 to 72, wherein the container is comprised in an overpouch, preferably in an overpouch being transparent and/or impermeable to oxygen.

### EXAMPLES

The following experiments were carried out in order to determine the optimal krill oil concentration.

### Examples 1a and 1b

The emulsions were prepared from the ingredients listed in table.

The oil phases were prepared by mixing fish oil extract (obtained from Solutex S.L.), krill oil (obtained from Olympic Seafood (Bioriginal Europe/Asia B.V.), the tocopherols and oleic acid. The mixture was heated to 55° C.

The aqueous phase was prepared by mixing water, glycerol and PEG. The mixture was heated to 60° C., and the pH was adjusted to 8.6 to 9.0.

The pre-emulsion was formed by adding the oil phase to the aqueous phase under continous agitation using a high shear mixer (Ultra Turrax T50).

The emulsion was formed by passing the pre-emulsion six times through a Niro Soavi Twin Panda 600 homogenizer at 500 bar and a temperature between 50 and 60° C.

Finally the emulsion was autoclaved at 121° C. for 15 minutes.

TABLE 1

| | Amount [g] | |
|---|---|---|
| Ingredient | Example 1a | Example 1b |
| Fish oil extract containing 670 mg/g EPA and | 10 | |
| 115 mg/g DHA | | |
| Krill oil (phospholipid content 40 wt. %) | 1.8 | 1.2 |

| | |
|---|---|
| Mixed tocopherols | 0.02 |
| Oleic acid | 0.15 |
| Glycerol | 2.25 |
| Polyethylen glycol | 1.0 |
| NaOH (1M) | to adjust pH to 8.6-9.0 |
| Water for injection | ad 100 |

The oil droplets of the emulsion according to example 1 a had a mean diameter of 156 nm when measured directly upon sterilization. The oil doplets of the emulsion according to example 1 b had a mean diameter of 167 nm when measured directly upon sterilization using an LS 13 320 Laser Diffraction Particle Size Analyser (Beckman Coulter) according to USP <729>.

Stability data for both emulsions are shown in table 3.

The results presented in table 3 show that both emulsion are stable for at least 12 weeks upon storage at 40° C.

It may thus be concluded that both 1.2% and 1.8% krill oil are suitable for obtaining stable emulsions for parenteral administration.

TABLE 2

| | | | | Mean | | | |
|---|---|---|---|---|---|---|---|
| | | | | droplet | | | |
| Storage | | | | diameter | | | |
| time | Storage | pH | | [nm] | | PFAT₅ | |
| [w] | T [° C.] | 1a | 1b | 1a | 1b | 1a | 1b |
| 0 | — | 9.04 | 8.80 | 156 | 167 | 0.027 | 0.022 |
| 1 | 25 | 9.04 | 8.79 | 151 | 173 | 0.023 | 0.017 |
| 2 | 25 | 8.99 | 8.66 | 159 | 169 | 0.028 | 0.019 |
| 4 | 40 | 8.91 | 8.58 | 152 | 166 | 0.032 | 0.023 |
| 12 | 40 | 8.85 | 8.40 | 153 | 161 | 0.049 | 0.048 |

### Examples 2a, 2b and 2c

The emulsions were prepared from the ingredients listed in table 3.

The oil phases were prepared by mixing soybean oil, MCT, olive oil, fish oil, krill oil (obtained from Olympic Seafood (Bioriginal Europe/Asia B.V.)) and alpha-tocopherol. The mixture was heated to 60° C.

The aqueous phase was prepared by mixing water, glycerol and sodium oleate. The mixture was heated to 60° C., and the pH was adjusted to 8.6 to 9.0.

The pre-emulsion was formed adding the oil phase to the aqueous phase under continous agitation using a high shear mixer (Ultra Turrax T50).

The emulsion was formed by passing the pre-emulsion six times through a Niro Soavi TwinPanda 600 homogenizer at 500 bar and a temperature between 50 and 60° C.

Finally the emulsion was autoclaved at 121° C. for 15 minutes.

TABLE 3

| | Amount [g] | | |
|---|---|---|---|
| Ingredient | Example 2a | Example 2b | Example 2c |
| Soybean oil | 6.0 | | 3.0 |
| MCT | 6.0 | | 3.0 |
| Olive oil | 5.0 | | 2.5 |
| Fish oil | 3.0 | | 1.5 |
| Krill oil (phospholipid content 40 | 1.2 | 2.4 | 1.2 |
| wt. %) | | | |

| | |
|---|---|
| Alpha-tocopherol | 0.02 |
| Glycerol | 2.25 |
| Sodium oleate | 0.03 |
| NaOH (1M) | to adjust pH to 8.6-9.0 |
| Water for injection | ad 100 |

The oil droplets of the emulsion according to example 2a had a mean diameter of 340 nm, the oil doplets of the emulsion according to example 2b had a mean diameter of 223 nm, and the oil droplets of the emulsion according to example 2c had a mean diameter of 205 nm when measured directly upon sterilization using an LS 13 320 Laser Diffraction Particle Size Analyser (Beckman Coulter) according to USP <729>.

Stability data for all three emulsions are shown in table 4.

From the results presented in table 4 it is apparent that the emulsions according to example 2a and 2c were stable for at least 4 week or 12 weeks respectively upon storage at 25 and 40° C.

The emulsion containing 2.4% krill oil, however, does not seem to be suitable for parenteral administration because the PFAT₅ value is exceeded already after 1 week of storage.

Thus, the optimal krill oil concentration may be concluded to be below 2.4%, irrespective of the lipid concentration.

TABLE 4

| | | | | | Mean | | | | | |
|---|---|---|---|---|---|---|---|---|---|---|
| Stor- | Stor- | | | | droplet | | | | | |
| age | age | | | | diameter | | | | | |
| time | T | pH | | | [nm] | | | PFAT₅ | | |
| [w] | [° C.] | 2a | 2b | 2c | 2a | 2b | 2c | 2a | 2b | 2c |
| 0 | — | 9.08 | 8.92 | 8.90 | 340 | 223 | 270 | 0.031 | 0.045 | 0.013 |
| 1 | 25 | 9.05 | 8.84 | 9.02 | 340 | 220 | 223 | 0.037 | 0.051 | 0.016 |
| | 40 | 9.04 | 9.05 | — | 346 | 223 | 266 | 0.038 | 0.056 | — |
| 2 | 25 | 9.07 | 8.82 | 8.96 | 340 | 212 | 268 | 0.049 | 0.065 | 0.016 |
| | 40 | — | — | — | — | — | — | — | — | — |
| 4 | 25 | 9.05 | 8.88 | 8.89 | 336 | 212 | 258 | 0.046 | 0.051 | 0.012 |
| | 40 | 8.75 | 8.68 | 8.71 | 342 | 217 | 253 | 0.039 | 0.055 | 0.016 |
| 12 | 25 | — | — | 8.71 | — | — | 273 | — | — | 0.018 |
| | 40 | — | — | 8.30 | — | — | 279 | — | — | 0.022 |

### Example 3

The emulsion was prepared from the ingredients listed in table 5.

The oil phase was prepared by mixing the purified fish oil and the krill oil (obtained from Olympic Seafood (Bioriginal Europe/Asia B.V.)). The mixture was heated to 60° C.

The aqueous phase was prepared by mixing water, glycerol and sodium oleate. The mixture was heated to 60° C., and the pH was adjusted to 8.6 to 9.0.

The pre-emulsion was formed by adding the oil phase to the aqueous phase under continous agitation using a high shear mixer (Ultra Turrax T50).

The emulsion was formed by passing the pre-emulsion six times through a Niro Soavi Twin Panda 600 homogenizer at 500 bar and a temperature between 50 and 60° C.

Finally the emulsion was autoclaved at 121° C. for 15 minutes.

TABLE 5

| Ingredient | Amount [g] |
|---|---|
| Purified fish oil | 10 |
| Krill oil (phospholipid content 40 wt. %) | 2.4 |
| Sodium oleate | 0.03 |
| Glycerol | 2.25 |
| NaOH (1M) | to adjust pH to 7.5-9.0 |
| Water for injection | ad 100 |

The oil droplets of the emulsion had a mean diameter of 164 nm when measured directly upon sterilization using a LS 13 320 Laser Diffraction Particle Size Analyser (Beckman Coulter) according to USP <729>.

Stability data for the emulsion are shown in table 6.

TABLE 6

| Storage time | Storage | | Mean droplet | |
|---|---|---|---|---|
| [weeks] | T [° C.] | pH | diameter [nm] | PFAT₅ |
| 0 | — | 8.77 | 164 | 0.037 |
| 1 | 25 | 8.77 | 162 | 0.031 |
| 2 | 25 | 8.50 | 164 | 0.037 |
| 4 | 25 | 8.48 | 157 | 0.037 |
| 12 | 25 | 8.33 | 163 | 0.054 |

The results presented in table 6 show that the emulsion according to example 3 does not seem to be suitable for parenteral administration because the PFAT₅ value is exceeded after 12 weeks of storage at 25° C.

Thus, the optimal krill oil concentration may be concluded to be below 2.4%.

### Examples 4a and 4b

The emulsions were prepared from the ingredients listed in table 7.

TABLE 7

| | Amount [g] | |
|---|---|---|
| Ingredient | Example 4a | Example 4b |
| Fish oil | | 10 |
| Soybean oil | 10 | |

| | |
|---|---|
| Krill oil (phospholipid content 40 wt. %) | 1.8 |
| Glycerol | 2.25 |
| NaOH (1M) | to adjust pH to 7.5-9.0 |
| Water for injection | ad 100 |

The oil phases were prepared by mixing either soybean oil or fish oil with the krill oil (obtained from Olympic Seafood (Bioriginal Europe/Asia B.V.)). The mixture was heated to 60° C.

The aqueous phase was prepared by mixing water and glycerol. The mixture was heated to 60° C., and the pH was adjusted to 7.5 to 9.0.

The pre-emulsion was formed adding the oil phase to the aqueous phase under continous agitation using a high shear mixer (Ultra Turrax T50).

The emulsion was formed by passing the pre-emulsion six times through a Niro Soavi Twin Panda 600 homogenizer at 500 bar and a temperature between 50 and 60° C.

Finally the emulsion was autoclaved at 121° C. for 15 minutes.

The oil droplets of the emulsion according to example 4a had a mean diameter of 193 nm and the oil doplets of the emulsion according to example 4b had a mean diameter of 161 nm when measured directly upon sterilization using a LS 13 320 Laser Diffraction Particle Size Analyser (Beckman Coulter) according to USP <729>.

Stability data for all three emulsions are shown in table 8.

TABLE 8

| | | | | Mean | | | |
|---|---|---|---|---|---|---|---|
| | | | | droplet | | | |
| Storage | | | | diameter | | | |
| time | Storage | pH | | [nm] | | PFAT₅ | |
| [w] | T [° C.] | 4a | 4b | 4a | 4b | 4a | 4b |
| 0 | — | 8.81 | 8.86 | 193 | 161 | 0.011 | 0.011 |
| 1 | 25 | 8.85 | 8.85 | 197 | 168 | 0.014 | 0.005 |
| 2 | 25 | — | 8.90 | — | 160 | — | 0.006 |
| 4 | 25 | 8.83 | 8.86 | 183 | 165 | 0.012 | 0.015 |

From the results presented in table 8 it is apparant that the emulsions according to examples 4a and 4b were stable for at least 4 weeks upon storage at 25° C.

It may thus be concluded that a concentration of 1.8% krill oil is suitable for obtaining stable emulsions for parenteral administration, even in the absence of a co-surfactant and a co-solvent.

1.-30. (canceled)

31. An oil-in-water emulsion comprising 0.5 to 2.2 wt % krill oil based on the total weight of the emulsion, wherein the emulsion is free of egg yolk lecithin and is formulated for parenteral administration.

32. The emulsion according to claim 31 comprising 2 to 30 wt % of an oil phase based on the total weight of the emulsion, wherein the oil phase comprises one or more oils selected from the group consisting of fish oil, fish oil extract, olive oil, soybean oil and MCT.

33. The emulsion according to claim 31, wherein the emulsion further comprises a pharmaceutically acceptable tonicity agent and an agent for pH adjustment.

34. The emulsion according to claim 31, wherein the emulsion further comprises at least one pharmaceutically acceptable antioxidant.

35. The emulsion according to claim 31, wherein the emulsion further comprises a pharmaceutically acceptable co-surfactant and/or co-solvent.

36. The emulsion according to claim 31, wherein the emulsion further comprises one or more of the following: glycerol as a pharmaceutically acceptable tonicity agent; sodium hydroxide (NaOH) for pH adjustment, an alpha, beta, gamma, or delta tocopherol as a pharmaceutically acceptable antioxidant; oleic acid or sodium oleate as a pharmaceutically acceptable co-surfactant; and polyethylene glycol (PEG) as a pharmaceutically acceptable co-solvent.

37. The emulsion according to claim 31, wherein oil droplets comprised in the oil-in-water emulsion have a mean diameter of 130 to 350 nm when measured directly upon sterilization of the emulsion.

38. A dose unit comprising 50 to 500 ml of the emulsion according to claim 31.

39. A method of treating or preventing malnutrition, stroke, sepsis, Alzheimer's disease, cancer, or a deficiency in essential fatty acids and/or EPA and DHA, the method comprising administering the oil-in-water emulsion according to claim 31 to a patient who is suffering from or who is at risk of malnutrition, stroke, sepsis, Alzheimer's disease, cancer, or a deficiency in essential fatty acids and/or EPA and DHA.

40. The method according to claim 39, wherein the patient is suffering from or is at risk of malnutrition and is at risk of stroke, sepsis, Alzheimer's disease or cancer.

41. The method of claim 39, wherein the patient is suffering from or at risk of malnutrition and a deficiency in essential fatty acids and/or EPA and DHA and is at risk of stroke, sepsis, Alzheimer's disease or cancer.

42. A method of providing parenteral nutrition, and/or supplementing essential fatty acids, and/or EPA and DHA to an individual, the method comprising administering the oil-in-water emulsion according to claim 31 to the individual.

43. The method of claim 42, wherein the individual is suffering from or is at risk of stroke, sepsis, Alzheimer's disease or cancer.

44. A container comprising the emulsion according to claim 31, wherein the container comprises glass or plastic material and is optionally configured as a bottle, a bag, or a syringe.

45. The container according to claim 44, wherein the plastic material comprises at least three layers.

46. The container according to claim 45, wherein at least one of the three layers comprises an inner layer that is in direct contact with the emulsion, the inner layer including a polyolefine co-polymer and a thermoplastic elastomer.

47. The container according to claim 45, wherein at least one of the three layers comprises an inner layer, the inner layer comprising 70 to 90 wt % of a polyolefine co-polymer and 10 to 30 wt % of a thermoplastic elastomer.

48. The container according to claim 46, wherein the thermoplastic elastomer comprises a styrenic block co-polymer.

49. The container according to claim 48, wherein the thermoplastic elastomer comprises Styrene-Ethylen-Butylen-Styrene.

50. The container according to claim 46, wherein the polyolefine co-polymer comprises a polypropylene-polyethylene co-polymer.

51. The container according to claim 45, wherein at least one of the three layers comprises an inner layer, the inner layer having a thickness of 10 to 90

52. A method for manufacturing an emulsion according to claim 31, comprising:
a) providing an oil phase comprising the krill oil and optionally one or more oils selected from the group consisting of fish oil, fish oil extract, olive oil, soy bean oil and MCT and/or at least one pharmaceutically acceptable antioxidant and/or a pharmaceutically acceptable co-surfactant;
b) providing an aqueous phase comprising water for injection and optionally a pharmaceutically acceptable tonicity agent and/or an agent for pH adjustment and/or a pharmaceutically acceptable co-surfactant and or a pharmaceutically acceptable co-solvent,
c) forming a pre-emulsion by mixing the oil phase provided in step a) with the aqueous phase provided in step b);
d) forming the emulsion by high-pressure homogenizing the pre-emulsion obtained in step c); and
e) sterilizing the emulsion obtained in step d), wherein optionally the emulsion is filled into a suitable container before or after sterilization.
